(19) United States
(12) Patent Application Publication
(10) Pub. No.: US 2003/0068416 A1
(43) Pub. Date: Apr. 10, 2003

(54) Method of lyophylization to reduce solvent content and enhance product recovery

(76) Inventors: Wilson Burgess, Clifton, VA (US); William N. Drohan, Springfield, VA (US); Martin J. MacPhee, Montgomery Village, MD (US); David M. Mann, Gaithersburg, MD (US); Dawson Beall, Gaithersburg, MD (US)

Correspondence Address: FLESHNER & KIM, LLP, P.O. Box 221200, Chantilly, VA, 20153-1200, US

(21) Appl. No.: 09/960,705

(22) Filed: Sep. 24, 2001

(51) Int. Cl. A23C 1/06; A61K 31/375

(52) U.S. Cl. 426/384; 426/385; 514/474

(57) ABSTRACT

The present invention relates to an improved method for lyophilization or freeze-drying. More specifically, the present invention relates to methods for reducing the residual solvent content of a material during lyophilization or freeze-drying by the addition of a compound effective therefor, such as ascorbic acid, or a suitable derivative thereof, such as a salt or ester thereof, or mannitol.

### BACKGROUND OF THE INVENTION

1. Field of the Invention

The present invention relates to an improved method for lyophilization or freeze-drying. More specifically, the present invention relates to methods for reducing the residual solvent content of a material during lyophilization or freeze-drying by the addition of a compound effective to reduce residual solvent content, such as mannitol, ascorbic acid or a suitable derivative of ascorbic acid, e.g., a salt or ester thereof.

2. Background of the Related Art

Lyophilization, also commonly referred to as freeze drying, is the process of removing solvent, such as water, from a product by sublimation and desorption. This process is performed in lyophilization equipment which consists of a drying chamber with temperature controlled shelves, a condenser to trap solvent removed from the product, a cooling system to supply refrigerant to the shelves and condenser, and a vacuum system to reduce the pressure in the chamber and condenser to facilitate the drying process.

Lyophilizers or freeze-driers can be supplied in a wide variety of sizes and configurations and can be equipped with options that allow system controls to range from fully manual to completely automated. For pharmaceutical compounds and biological materials that undergo hydrolytic degradation, lyophilization offers a means of improving their stability and shelf life. Many parenteral medications such as vaccines, proteins, peptides, and antibiotics have been successfully lyophilized. New biotechnology products will also increase the demand for freeze drying equipment and processes.

Early attempts at lyophilization were largely empirical in nature because the process variables were not thoroughly understood. Much of the “black magic” of freeze drying, however, has been replaced through basic research over the last twenty years. Lyophilization equipment and control mechanisms continue to evolve, based on scientific evaluation of thermal, physical and chemical data derived from freeze drying cycles and products.

Lyophilization cycles may include three phases: freezing (thermal treatment), primary drying (sublimation), and secondary drying (desorption). Conditions in the freeze dryer are varied through the cycle to insure that the resulting product has the desired physical and chemical properties, and that the required stability is achieved.

During the freezing phase, (thermal treatment) the goal is to freeze the mobile solvent, generally water, of the product. Significant supercooling may be encountered, so the product temperature may have to be much lower than the actual freezing point of the solution before freezing occurs. The rate of cooling will influence the structure of the frozen solvent matrix. If the solvent freezes quickly, the solvent crystals will be small. This may cause a finer pore structure in the product with higher resistance to flow of solvent vapor and longer primary drying time. If freezing is slower, ice crystals will grow from the cooling surface and may be larger. The resultant product may have courser pore structure and perhaps a shorter primary drying time.

The method of cooling will also effect the structure and appearance of the matrix and final product. If the solution is frozen in vials on the cooled shelf, solvent crystals will grow from the bottom of the vial toward the top, while immersion in a cooling fluid will cause crystal growth from the bottom and sides of the vial. Because some materials form glassy layers, cooling conditions must be controlled to avoid the formation of the dense “skin” on the surface of the frozen product that may impede the escape of solvent vapor during subsequent drying phases.

A term that is frequently encountered in discussions about freeze drying is eutectic point. On a phase diagram, this is the temperature and composition coordinate below which only the solid phase exists. It should be understood that, depending on the composition of the solution, there may be more than one eutectic point for a product or none at all. During the freezing phase, the product must be cooled to a temperature below its lowest eutectic point. This temperature may then be maintained throughout the primary drying phase.

It should be noted that products will not necessarily have a eutectic point. For products with components that do not crystallize during freezing, drying should be performed at temperatures below the glass transition temperature of the amorphous phase (multicomponent mixture). The glass transition temperature will be determined by the composition of the amorphous phase in the frozen product, which, in turn, is dictated by the product formulation and the freezing procedure employed. Mannitol and some other compounds can exist as an amorphous phase or exhibit a crystalline phase depending upon its thermal history.

In the primary drying phase, the chamber pressure is reduced, and heat is applied to the product to cause the frozen mobile solvent to sublime. The solvent vapor is collected on the surface of a condenser. The condenser must have sufficient surface area and cooling capacity to hold all of the sublimed solvent from the batch at a temperature lower than the product temperature. If the temperature of the frozen solvent on the condenser is warmer than the product, solvent vapor will tend to move toward the product, and drying will stop.

It is important to control the drying rate and the heating rate during this phase. If the drying proceeds too rapidly, the dried product can be blown out of the container by escaping solvent vapor and lost. If the product is heated too rapidly, it will melt or collapse. This may cause degradation of the product, and will certainly change the physical characteristics of the dried material, making it visually unappealing and harder to reconstitute. While frozen mobile solvent is present, the product must be held below the eutectic temperature or glass transition temperature.

As the solvent sublimes, the product on its own would cool; however, within a freeze-drier, the temperature differential between the product and shelf results in a slow rise in the temperature of the product. This is because the shelf is supplying the heat of sublimation.

Many modern drying cycles use chamber pressure control to control drying rate. At very low pressures, the main form of heat transfer is conduction from the shelf through the bottom of the product container. Since glass is an insulator, this process is not very efficient, and drying can be slow. To improve the heat transfer mechanism, inert gas such as nitrogen may be introduced into the drying chamber at a controlled rate. The presence of these gas molecules facilitates heating of the walls of the container in addition to conduction through the bottom of the container, thereby increasing the amount of heat being supplied to the product per unit time. This will enhance the drying rate, reduce the cycle time, and reduce energy and labor costs associated with a lengthy process.

If the pressure in the chamber exceeds the solvent vapor pressure of the product, however, the solvent may not be able to sublime. All of the energy from the heat source will be used to increase the product temperature until melting occurs. Therefore, the accuracy and precision of the pressure control system are critical to successful lyophilization.

Since there is no mobile solvent, such as water, in the product at the end of primary drying, the shelf temperature may be increased without causing melting. Therefore, temperature is increased to desorb bound solvent, such as water of crystallization, until the residual solvent content falls to the range required for optimum product stability. This phase is referred to as secondary drying, and is usually performed at the maximum vacuum the dryer can achieve, although there are products that benefit from increased pressures, too. One must be careful not to increase product temperatures too fast, however, so as not to exceed the glass transition of some products. Products containing ¹⁰% or less water can still collapse if this temperature is exceeded.

The length of the secondary drying phase will be determined by the product. Many products, such as proteins and peptides, require some water to maintain their secondary and tertiary structure. If this water is removed, the material may be denatured and lose some or all of its desired activity. In such cases, the final residual solvent content of the product must be carefully controlled. In addition, excessive heat may cause the dried product to char or shrink.

Samples of the product being lyophylized may be taken from the lyophylizer during the lyophylization process to determine the residual solvent content at various stages of the lyophylization cycle. This is accomplished by the use of a sample remover, known to those skilled in the art as a ‘thief’. Analysis of these ‘thiefed’ samples can be used to optimize the cycle and determine the best combination of temperatures, pressures and times to accomplish the lyophylization as desired.

There is therefore a need for improved lyophilization processes, whereby the residual solvent content of a product can be reduced without the need for the application of additional heat and/or vacuum.

### SUMMARY OF THE INVENTION

An object of the invention is to solve at least the above problems and/or disadvantages and to provide at least the advantages described hereinafter.

Accordingly, it is an object of the present invention to provide improved methods of lyophilizing or freeze-drying a product. Other objects, features and advantages of the present invention will be set forth in the detailed description of preferred embodiments that follows, and in part will be apparent from the description or may be learned by practice of the invention. These objects and advantages of the invention will be realized and attained by the methods particularly pointed out in the written description and claims hereof.

In accordance with these and other objects, a first embodiment of the present invention is directed to an improved method for lyophilizing or freeze-drying a product, wherein the improvement comprises adding ascorbate to the product prior to lyophilizing or freeze-drying the product, wherein the amount of ascorbate added is effective to lower the residual solvent content of the product following lyophilization or freeze-drying.

In accordance with these and other objects, a second embodiment of the present invention is directed to an improved method for lyophilizing or freeze-drying a product, wherein the improvement comprises adding a compound effective to reduce residual solvent content to the product prior to lyophilizing or freeze-drying the product, wherein the amount of a compound effective to reduce residual solvent content added is effective to lower the residual solvent content of the product following lyophilization or freeze-drying.

Another embodiment of the present invention is directed to a composition of matter comprising a product that has been subjected to lyophilization or freeze-drying in the presence of an effective amount of ascorbate.

A further embodiment of the present invention is directed to a product that has been subjected to lyophilization or freeze-drying in the presence of an effective amount of a compound effective to reduce residual solvent content.

Additional advantages, including increased solubility and buffering, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### 1. DETAILED DESCRIPTION OF THE PREFERRED EMBODIMENTS

A. Definitions

Unless defined otherwise, all technical and scientific terms used herein are intended to have the same meaning as is commonly understood by one of ordinary skill in the relevant art.

As used herein, the singular forms “a,” “an” and “the” include the plural reference unless the context clearly dictates otherwise.

As used herein, the terms “lyophilization” and “freeze-drying” are used interchangeably to mean a process by which a desired product containing a solvent, such as water, is cooled to a sufficient temperature at which some or all of the solvent is frozen and then the frozen solvent is removed by one or two drying steps, a primary drying step (which involves removal of unbound solvent by sublimation) and, frequently, a subsequent secondary drying step (which involves removal of bound solvent by desorption). In some lyophylization cycles the temperature is ramped in a constant or step-wise fashion up from a low temperature to a higher temperature. In this case, the primary drying cycle corresponds to the low temperature portion of the cycle and the secondary drying cycle is the portion conducted at higher temperatures (ie at temperatures above the melting or eutectic point of the product).

As used herein, the terms “lyophilizer” and “freeze-dryer” are used interchangeably to mean the equipment in which a lyophilization or freeze-drying process may take place. During these processes, the lyophilizer or freeze-dryer performs two important functions: (i) providing a safe environment for the product whereby the environment is free of sources of product contamination, e.g., particulate matter including, but not limited to, dust, lint, glass, metal, hair and oxides, harmful vapors, including, but not limited to, vapors from cleaning solution, sterilization liquid or fluids and gases used in the operation of the freeze-dryer, and microorganisms; and (ii) providing the requisite operating parameters to conduct the drying processes, e.g., pressure and temperature, which are generally established by the operator.

As known to those skilled in the art, a conventional lyophilizer or freeze-dryer generally contains the following components:

(1) a drying chamber to provide a thermally insulated environment usually containing shelves and means for loading and unloading product from the shelves, and frequently a means of closing the product containers while within the chamber;

(2) a condenser to remove the solvent vapor that is either sublimated or desorbed from the product. Desorption or sublimation is accomplished by directing the flow of the vapors from the product over a surface. An internal condenser is one in which the condensing surfaces are located in the drying chamber whereas with an external condenser, the refrigeration surfaces are contained in a separate insulated chamber. The principle advantages of the internal condenser are reduced space requirements and reduced cost. The principle advantage of the external condenser is that the condenser can be isolated from the drying chamber during the backfilling of the chamber with a dry gas such as nitrogen. By isolating the condenser during the backfilling of the drying chamber, the possibility of the transport of solvent vapor from the condensed ice surface back to the product is eliminated. If a condenser is not used to trap the solvent vapor, then phosphorous pentoxide may be employed for this purpose;

(3) a vacuum system which is generally a mechanical pumping system that removes the non-condensable gases. Pumps may be either oil sealed or dry. With oil sealed mechanical pumps, one must be careful to operate the dryer so that no hydrocarbon vapors from the pump can “backstream” into the drying chamber. There are pumps that can operate in the absence of an oil seal; and

(4) instrumentation, such as a vacuum gauge (which determines the pressure during the drying processes) and temperature sensors (which measure the shelf and product temperatures throughout the process). Often, lyophilizers or freeze-dryers are equipped with a computer system that will control the pressure in the drying chamber and shelf temperature as a function of time.

As used herein, the term “residual solvent content” is intended to mean the amount or proportion of liquid in a product, and includes both freely-available solvent and bound solvent. Freely-available solvent is the solvent, such as water or an organic solvent (e.g. ethanol, isopropanol, acetone, polyethylene glycol, etc.), present in the product that is not bound to or complexed with one or more of the non-liquid components of the product undergoing lyophilization or freeze-drying and includes intracellular water.

When the solvent is water, the residual solvent content referenced herein refer to levels determined by the FDA approved, modified Karl Fischer method (Meyer and Boyd, Analytical Chem., 31:215-219, 1959; May, et al., J. Biol. Standardization, 10:249-259, 1982; Centers for Biologics Evaluation and Research, FDA, Docket No. 89D-0140, 83-93; 1990) or by near infrared spectroscopy.

Quantitation of the residual levels of other solvents may be determined by means well known in the art, depending upon which solvent is employed. The proportion of residual solvent to solute may also be considered to be a reflection of the concentration of the solute within the solvent. When so expressed, the greater the concentration of the solute, the lower the amount of residual solvent.

As used herein, the term “product” (alone and within the phrase “product formulation”) is intended to mean any substance that can be subjected to lyophilization or freeze drying, and includes products derived or obtained from a living or non-living organism. Illustrative examples of products derived from a living or non-living organism include, but are not limited to, the following: cells; tissues; blood or blood components; proteins, including recombinant and transgenic proteins, and proetinaceous materials; enzymes, including digestive enzymes, such as trypsin, chymotrypsin, alpha-galactosidase and iduronodate-2-sulfatase; immunoglobulins, including mono and polyimmunoglobulins; botanicals; food and the like. Preferred examples of products include, but are not limited to, the following: ligaments; tendons; nerves; bone, including demineralized bone matrix, grafts, joints, femurs, femoral heads, etc.; teeth; skin grafts; bone marrow, including bone marrow cell suspensions, whole or processed; heart valves; cartilage; corneas; arteries and veins; organs, including organs for transplantation, such as hearts, livers, lungs, kidneys, intestines, pancreas, limbs and digits; lipids; carbohydrates; collagen, including native, afibrillar, atelomeric, soluble and insoluble, recombinant and transgenic, both native sequence and modified; chitin, chitosan and its derivatives, including NO-carboxy chitosan (NOCC); stem cells, islet of Langerhans cells and other cells for transplantation, including genetically altered cells; red blood cells; white blood cells, including monocytes; and platelets.

As used herein, the term “blood components” is intended to mean one or more of the components that may be separated from whole blood and include, but are not limited to, the following: cellular blood components, such as red blood cells, white blood cells and platelets; blood proteins, such as blood clotting factors, including thrombin, prothrombin and factor XIII, enzymes, albumin, plasminogen, fibrinogen and immunoglobulins; and liquid blood components, such as plasma, cryoprecipitate, plasma fractions, including plasma protein fraction (PPF), and plasma-containing compositions.

As used herein, the term “cellular blood component” is intended to mean one or more of the components of whole blood that comprises cells, such as red blood cells, white blood cells, stem cells and platelets.

As used herein, the term “blood protein” is intended to mean one or more of the proteins that are normally found in whole blood. Illustrative examples of blood proteins found in mammals, including humans, include, but are not limited to, the following: coagulation proteins, both vitamin I-dependent, such as Factor VII and Factor IX, and non-vitamin K-dependent, such as Factor VIII and von Willebrands factor; albumin; lipoproteins, including high density lipoproteins and low density lipoproteins; complement proteins; globulins, such as immunoglobulins IgA, IgM, IgG and IgE; and the like. A preferred group of blood proteins includes Factor I (fibrinogen), Factor II (prothrombin), Factor III (tissue factor), Factor V (proaccelerin), Factor VI (accelerin), Factor VII (proconvertin, serum prothrombin conversion), Factor VIII (antihemophiliac factor A), Factor IX (antihemophiliac factor B), Factor X (Stuart-Prower factor), Factor XI (plasma thromboplastin antecedent), Factor XII (Hageman factor), Factor XIII (protransglutamidase), von Willebrands factor (vWF), Factor Ia, Factor IIa, Factor IIIa, Factor Va, Factor VIa, Factor VIIa, Factor VIIIa, Factor IXa, Factor Xa, Factor XIa, Factor XIIa and Factor XIIIa. Another preferred group of blood proteins includes proteins found inside red blood cells, such as hemoglobin and various growth factors, and derivatives of these proteins. Yet another preferred group of blood proteins include proteins found in commercially available plasma protein fraction products, such as Plasma-Plex® (Centeon/Aventis Behring), Protenate® (Baxter Laboratories), Plasmanate® (Bayer Biological) and Plasmatein® (Alpha Therapeutic)

As used herein, the term “liquid blood component” is intended to mean one or more portion of the whole blood of humans or animals (as found prior to coagulation) and serum (the fluid, non-cellular portion of the whole blood of humans or animals as found after coagulation).

As used herein, the term “a biologically compatible solution” is intended to mean a solution to which a biological material may be exposed, such as by being suspended or dissolved therein, and remain viable, i.e., retain its essential biological and physiological characteristics.

As used herein, the term “a biologically compatible buffered solution” is intended to mean a biologically compatible solution having a pH and osmotic properties (e.g., tonicity, osmolality and/or oncotic pressure) suitable for maintaining the integrity of the material(s) therein. Suitable biologically compatible buffered solutions typically have a pH between 4 and 8.5 and are isotonic or only moderately hypotonic or hypertonic. Biologically compatible buffered solutions ate known and readily available to those of skill in the art.

As used herein, the term “proteinaceous material” is intended to mean any material derived or obtained from a living organism that comprises at least one protein or peptide. A proteinaceous material may be a naturally occurring material, either in its native state or following processing/purification and/or derivatization, or an artificially produced material, produced by chemical synthesis or recombinant/transgenic technology and, optionally, process/purified and/or derivatized. Illustrative examples of proteinaceous materials include, but are not limited to, the following: proteins and peptides produced from cell culture; milk and other dairy products; ascites; hormones; growth factors; materials, including pharmaceuticals, extracted or isolated from animal tissue, such as heparin and insulin, or plant matter; plasma, including fresh, frozen and freeze-dried, and plasma protein fraction; fibrinogen and derivatives thereof, fibrin, fibrin I, fibrin II, soluble fibrin and fibrin monomer, and/or fibrin sealant products; whole blood; protein C; protein S; alpha-1 anti-trypsin (alpha-1 protease inhibitor); butyl-cholinesterase; anticoagulants, such as streptokinase; tissue plasminogen activator (tPA); erythropoietin (EPO); urokinase; Neupogen™; anti-thrombin-3; alpha-galactosidase; (fetal) bovine serum/horse serum; meat; immunoglobulins, including anti-sera, monoclonal antibodies, polyclonal antibodies and genetically engineered or produced antibodies; albumin; alpha-globulins; beta-globulins; gamma-globulins; coagulation proteins; complement proteins; and interferons.

As used herein, the term “ascorbate” is intended to mean ascorbic acid or a derivative thereof, including salts of ascorbic acid, such as the sodium salt (sodium ascorbate), and esters of ascorbic acid, such as the methyl ester (methyl ascorbate).

As used herein, the term “a compound effective to reduce residual solvent content” is intended to mean a compound which, when employed in the process described herein in an effective amount, reduces residual solvent content of products produced thereby. Such compounds include mannitol, ascorbic acid or a suitable derivative of ascorbic acid, e.g., a salt or ester thereof.

B. Particularly Preferred Embodiments

A first preferred embodiment of the present invention is directed to an improved method for lyophilizing or freeze-drying a product, wherein the improvement comprises adding ascorbate to the product prior to lyophilizing or freeze-drying the product, wherein the amount of ascorbate added is effective to lower the residual solvent content of the product following lyophilization or freeze-drying.

The basic methods and techniques of lyophilization and freeze-drying are well-known to those skilled in the art. In general, the solvent-containing product is first subjected to a sufficient temperature until some or all of the solvent is frozen (thermal treatment). The frozen solvent-containing product is then subjected to a one or two-step drying process.

During the first step, which is also known as primary drying, the frozen solvent-containing product is subjected to a sufficient temperature and pressure such that the frozen freely-available solvent undergoes sublimation.

During the second step, which is often employed, and known as secondary drying, the product is subjected to sufficient temperature and pressure such that the bound solvent undergoes desorption.

Following desorption, the product may be sealed in a container, generally under vacuum or in an inert gas, and stored at ambient temperature. Frequently, the initial seal is applied within the lyophylizer itself. The product may also subsequently be refrigerated or frozen. Suitable methods of storage may vary from product to product and can be readily determined empirically by the skilled artisan.

Similarly, an appropriate storage container can be readily determined by the skilled artisan. Suitable containers are well-known in the art and include, but are not limited to, vials, tubes and ampules, either capped or uncapped (wherein the cap may contain an immobilized desiccant). The container and/or cap may be completely or partially covered by a sheath or film comprised of any suitable material, such as plastic or Mylar®. Such a sheath or film may prevent leakage and/or deterioration of the container contents.

The container may be constructed of any suitable material, such as plastic or glass, that is not deleterious to the product undergoing lyophilization (or freeze-drying) and storage. Similarly, the cap may be constructed of any suitable material, such as plastic or rubber, that is not deleterious to the product undergoing lyophilization or freeze-drying.

The lyophilized or freeze-dried product of the invention may be employed in a pharmaceutical composition and dosage form and thus, may comprise a pharmaceutically acceptable carrier such as an excipient or diluent. In addition, suitable carriers such as binders, fillers, disintegrants, lubricants, cellulose, starches, sugars, granulating agents, etc. may be added to the product prior to, during or after lyophilization or freeze-drying. Such additives may be present in a concentration ranging from about 0.001 to about 99.999% w/v.

The concentration of a compound effective to reduce residual solvent content, such as ascorbate or mannitol, in the product formulation is that amount sufficient to achieve the desired residual solvent content. The range of effective concentrations can be easily determined by one having ordinary skill in the art by varying the amount of a compound effective to reduce residual solvent content to achieve the desired residual solvent content. Typical concentrations of ascorbate, for instance, in the product formulation ranges from about 5 to about 500 mM, more preferably, from about 10 to about 400 mM, even more preferably, from about 50 to about 300 mM, and most preferably, from about 75 to about 200 mM. Most preferably, the concentration of ascorbate is about 100 mM.

One or more cryoprotectants may also be present in the product if desired. The selection of a particular cryoprotectant is well-within the purview of the skilled artisan and may be determined empirically. Examples of suitable cryoprotectants include, but are not limited to, carbohydrates, lipophilic molecules (such as sterols and glycols) linked to molecules containing polyhydroxyl groups (such as carbohydrates) via hydrophilic groups (such as polyoxyethylene), blood, glycerol compounds, propanediol, dimethyl sulfoxide (DMSO), sulfite compounds, butylated hydroxy anisole, buytlated hydroxy toluene, cystein, cysteinate HCl, dithionite sodium, gentisic acid compounds, glutamate monosodium, formaldehyde sulfoxylate sodium, propyl gallate, thioglycoate sodium, ethanol, acetone, etc. See, e.g., U.S. Pat. Nos. 4,931,361, 5,856,172, 6,060,233 and 6,258,821.

The solvent employed in the present invention may be an aqueous solvent, a non-aqueous solvent or a mixture of aqueous and/or non-aqueous solvents. For example, the solvent may be a non-aqueous solvent combined with water. If a non-aqueous solvent is employed, then, preferably, the non-aqueous solvent is not prone to the formation of free radicals when irradiated, and most preferably, the non-aqueous solvent is not prone to the formation of free radicals when irradiated and has little or no dissolved oxygen or other gas(es) that is (are) prone to the formation of free radicals on irradiation. Volatile solvents are also preferred. Exemplary solvents include, but are not limited to, water, including, but not limited to, sterile water, saline, alcohols, dextrose in water, benzyl benzoate, cottonseed oil, N,N-dimethylacetamide, glycerin, glyerol, peanut oil, culture media, polyethylene glycol, poppyseed oil, propylene glycol, safflower oil, sesame oil, soybean oil and vegetable oil. Other solvents, solvent mixtures or solvent systems are well-known and a suitable solvent or solvent mixture may be determined empirically by one skilled in the art.

When the solvent is water, the preferred residual solvent content is generally less than about 15%, typically less than about 10%, usually less than about 5%, preferably less than about 3.0%, more preferably less than about 2.0%, even more preferably less than about 1.0%, still more preferably less than about 0.5%, still even more preferably less than about 0.2% and most preferably less than about 0.08%.

In certain embodiments of the present invention, the residual solvent content of a particular product may be found to lie within a range, rather than at a specific point. Such a range for the preferred residual solvent content of a particular biological material, for example, may be determined empirically by one skilled in the art.

It has recently been shown that the reduction of residual solvent content in biological material by lyophylization can result in a composition that can be sterilized by irradiation without an unacceptable loss of desired biological activity. In certain embodiments of the present invention, the addition to a biological material of a compound, such as ascorbate or mannitol, followed by lyophylization, results in a composition that is suitable for sterilization by irradiation.

### EXAMPLES

The following examples are illustrative, but not limiting, of the present invention. Other suitable modifications and adaptations ate of the variety normally encountered by those skilled in the art and are fully within the spirit and scope of the present invention.

### Example 1

In this experiment, trypsin was lyophilized alone or in the presence of sodium ascorbate.

Method

1 ml aliquots of trypsin alone or with 100 mM sodium ascorbate were placed in 3 ml vials. Samples were prepared in triplicate and subjected to lyophilization (primary and secondary drying cycles; 62 hours).

| | Thermal | Primary | Secondary | Shelf Temp. | Time | Temp. | Chamber |
|---|---|---|---|---|---|---|---|
| Step | Treatment | Drying | Drying | (° C.) | (minutes) | Control | Press. (mT) |
| 1 | ✓ | | | −50 | 120 | Rate | Atmospheric |
| | | | | | | | pressure |
| 2 | ✓ | | | −50 | 360 | Hold | Atmospheric |
| | | | | | | | pressure |
| 3 | | ✓ | | −40 | 60 | Hold | 100 |
| 4 | | ✓ | | −25 | 75 | Rate | 100 |
| 5 | | ✓ | | −25 | 360 | Hold | 100 |
| 6 | | ✓ | | 35 | 240 | Rate | 100 |
| 7 | | | ✓ | 35 | 1080 | Hold | 10 |
| 8 | | | ✓ | 40 | 1425 | Hold | 10 |

Results

In the absence of sodium ascorbate, lyophilized trypsin exhibited a residual solvent content of about 1.8% water. In the presence of sodium ascorbate, however, lyophilized trypsin exhibited a residual solvent content of about 0.7% water.

### Example 2

In this experiment, trypsin was lyophilized alone or in the presence of sodium ascorbate.

Method

1 ml aliquots of trypsin alone or with 100 mM sodium ascorbate were placed in 3 ml vials. Samples were prepared in triplicate and subjected to lyophilization. Samples were removed (using sample thief) at the end of primary drying (22 hours after start of the cycle), the cycle continued to completion (62 hours after start of cycle) at this time vials were sealed under vacuum and removed from the freeze dryer.

| | Thermal | Primary | Secondary | Shelf Temp. | Time | Temp. | Chamber |
|---|---|---|---|---|---|---|---|
| Step | Treatment | Drying | Drying | (° C.) | (minutes) | Control | Press. (mT) |
| 1 | ✓ | | | −50 | 120 | Rate | Atmospheric |
| | | | | | | | pressure |
| 2 | ✓ | | | −50 | 360 | Hold | Atmospheric |
| | | | | | | | pressure |
| 3 | | ✓ | | −40 | 60 | Hold | 100 |
| 4 | | ✓ | | −25 | 75 | Rate | 100 |
| 5 | | ✓ | | −25 | 360 | hold | 100 |
| 6 | | ✓ | | 35 | 240 | rate | 100 |
| 7 | | | ✓ | 35 | 1080 | Hold | 10 |
| 8 | | | ✓ | 40 | 1425 | Hold | 10 |

Results

In the absence of sodium ascorbate, lyophilized trypsin exhibited a residual solvent content of about 5.8% water following a primary drying cycle alone and a residual solvent content of about 5.4% water following a combination of a primary and a secondary drying cycle.

In the presence of sodium ascorbate, however, lyophilized trypsin exhibited a residual solvent content of about 2.8% water following a primary drying cycle alone and a residual solvent content of about 1.1% water following a combination of a primary and a secondary drying cycle.

### Example 3

In this experiment, trypsin was lyophilized alone or in the presence of sodium ascorbate.

Method

1 ml aliquots of trypsin alone or with 100 mM sodium ascorbate were placed in 3 ml vials and frozen overnight at −70° C. Samples were prepared in quadruplicate and subjected to lyophilization. Samples were removed (using sample thief) at the end of primary drying (22 hours after start of the cycle), the cycle continued to completion (62 hours after start of cycle) at this time vials were sealed under vacuum and removed from the freeze dryer.

| | Thermal | Primary | Secondary | Shelf Temp. | Time | Temp. | Chamber |
|---|---|---|---|---|---|---|---|
| Step | Treatment | Drying | Drying | (° C.) | (minutes) | Control | Press. (mT) |
| 1 | ✓ | | | −50 | 120 | Rate | Atmospheric |
| | | | | | | | pressure |
| 2 | ✓ | | | −50 | 360 | Hold | Atmospheric |
| | | | | | | | pressure |
| 3 | | ✓ | | −40 | 60 | Hold | 100 |
| 4 | | ✓ | | −25 | 75 | Rate | 100 |
| 5 | | ✓ | | −25 | 360 | hold | 100 |
| 6 | | ✓ | | 35 | 240 | rate | 100 |
| 7 | | | ✓ | 35 | 1080 | Hold | 10 |
| 8 | | | ✓ | 40 | 1425 | Hold | 10 |

Results

In the absence of ascorbate, lyophilized trypsin exhibited a residual solvent content of about 3.9% water. In the presence of ascorbate, however, lyophilized trypsin prepared under identical conditions exhibited a residual solvent content of only 0.7% water.

### Example 4

In this experiment, a monoclonal antibody preparation was lyophilized alone or in the presence of sodium ascorbate.

Method

1 ml aliquots of sample (either 100 ug anti-insulin mouse antibody and 10 mg BSA (1%) or 100 ug anti-insulin mouse antibody, 10 mg BSA (1%) and 19.8 mg ascorbate (100 mM)) were placed in 3 ml vials. Samples (in duplicate) were subjected to freeze-drying,(see Table below for the cycle conditions). Samples were removed (using sample thief at the end of primary drying (22 hours after start of the cycle), the cycle continued to completion (62 hours after start of cycle) at this time vials were sealed closed under vacuum and removed from the freeze dryer.

| | Thermal | Primary | Secondary | Shelf Temp. | Time | Temp. | Chamber |
|---|---|---|---|---|---|---|---|
| Step | Treatment | Drying | Drying | (° C.) | (minutes) | Control | Press. (mT) |
| 1 | ✓ | | | −50 | 120 | Rate | Atmospheric |
| | | | | | | | pressure |
| 2 | ✓ | | | −50 | 360 | Hold | Atmospheric |
| | | | | | | | pressure |
| 3 | | ✓ | | −40 | 60 | Hold | 100 |
| 4 | | ✓ | | −25 | 75 | Rate | 100 |
| 5 | | ✓ | | −25 | 360 | hold | 100 |
| 6 | | ✓ | | 35 | 240 | rate | 100 |
| 7 | | | ✓ | 35 | 1080 | Hold | 10 |
| 8 | | | ✓ | 40 | 1425 | Hold | 10 |

Results

In the absence of ascorbate, lyophilized anti-insulin mouse antibody exhibited a residual solvent content of about 4.05% water after a primary drying cycle alone and a residual solvent content of about 3.89% water after a combination of a primary and a secondary drying cycle.

In the presence of ascorbate, however, lyophilized anti-insulin mouse antibody exhibited a residual solvent content of about 3.6% water after a primary drying cycle alone and a residual solvent content of about 1.0% water after a combination of a primary and a secondary drying cycle.

### Example 5

In this experiment, two different proteins were subjected to freeze-drying in the presence or absence of 100 mM sodium ascorbate and/or mannitol.

Method

Samples of human IgG and Albumin were each aliquoted into 3 ml Wheaton vials and partially stoppered with Stelmi stoppers. The vials were placed onto aluminum freeze-drying trays and placed into the freeze-dryer. Three independent freeze-drying runs were conducted for each protein. At the completion of each freeze-drying run, the vials were stoppered and sealed with aluminum seals until analyzed. The freeze-drying conditions are indicated in the table below:

| | Thermal | Primary | Secondary | Shelf Temp. | Time | Temp. | Chamber |
|---|---|---|---|---|---|---|---|
| Step | Treatment | Drying | Drying | (° C.) | (minutes) | Control | Press. (mT) |
| 1 | ✓ | | | −50 | 180 | Rate | Atmospheric |
| | | | | | | | pressure |
| 2 | ✓ | | | −50 | 300 | Hold | Atmospheric |
| | | | | | | | pressure |
| 3 | | ✓ | | −40 | 60 | Hold | 100 |
| 4 | | ✓ | | −25 | 150 | Rate | 100 |
| 5 | | ✓ | | −25 | 360 | Hold | 100 |
| 6 | | ✓ | | 35 | 360 | Rate | 100 |
| 7 | | | ✓ | 35 | 720 | Hold | 30 |
| 8 | | | ✓ | 40 | 1440 | Hold | 10 |
| 9 | | | ✓ | 40 | 1440 | Hold | 0 |

Solutions

Human IgG—1 ml of reprocessed human IgG (10%) was added to vials. Additional vials contained (1) IgG+10% mannitol, (2) IgG+100 mM sodium ascorbate and (3) IgG+10% mannitol+100 mM sodium ascorbate.

Albumin—1 ml of either 12.5% or 25% Albumin was added to vials. Additional vials contained (1) Albumin+10% mannitol, (2) Albumin+100 mM sodium ascorbate and (3) Albumin+10% mannitol+100 mM sodium ascorbate.

Results

In the absence of ascorbate, lyophilized human IgG exhibited a residual solvent content of about 1.0% water. In the presence of 100 mM sodium ascorbate, however, lyophilized human IgG exhibited a residual solvent content of about 0.55% water. In the presence of mannitol, the residual solvent content was 0.3% water. The combination of ascorbate and mannitol further reduced the residual moisture content to 0.2%

In the absence of ascorbate, a lyophilized 12.5% albumin preparation exhibited a residual solvent content of about 1.25% water. In the presence of sodium ascorbate, however, a lyophilized 12.5% albumin preparation prepared under identical conditions exhibited a residual solvent content of only about 0.75% water. In the presence of mannitol, the residual solvent content was 0.3% water. The combination of ascorbate and mannitol reduced the residual moisture content to 0.3%

In the absence of ascorbate, a lyophilized 25% albumin preparation exhibited a residual solvent content of about 0.55% water. In the presence of sodium ascorbate, a lyophilized 25% albumin preparation prepared under identical conditions exhibited a residual solvent content of only about 0.5% water. In the presence of mannitol, the residual solvent content was 0.35% water. The combination of ascorbate and mannitol further reduced the residual moisture content to 0.3%

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

The foregoing embodiments and advantages are merely exemplary and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art.

What is claimed is:

1. An improved method for lyophilizing or freeze-drying a product, wherein the improvement comprises adding ascorbate to the product prior to lyophilizing or freeze-drying the product, the amount of said ascorbate added being effective to lower the residual solvent content of said product following lyophilization or freeze-drying.

2. The method according to claim 1, wherein the product has a residual solvent content of less than about 15% following said lyophilization or freeze-drying.

3. The method according to claim 1, wherein the product has a residual solvent content of less than about 10% following said lyophilization or freeze-drying.

4. The method according to claim 1, wherein the product has a residual solvent content of less than about 5% following said lyophilization or freeze-drying.

5. The method according to claim 1, wherein the product has a residual solvent content of less than about 3.0% following said lyophilization or freeze-drying.

6. The method according to claim 1, wherein the product has a residual solvent content of less than about 2.0% following said lyophilization or freeze-drying.

7. The method according to claim 1, wherein the product has a residual solvent content of less than about 1.0% following said lyophilization or freeze-drying.

8. The method according to claim 1, wherein the product has a residual solvent content of less than about 0.5% following said lyophilization or freeze-drying.

9. The method according to claim 1, wherein the product has a residual solvent content of less than about 0.2% following said lyophilization or freeze-drying.

10. The method according to claim 1, wherein the product has a residual solvent content of less than about 0.08% following said lyophilization or freeze-drying.

11. The method according to claim 1, wherein said ascorbate is sodium ascorbate.

12. The method according to claim 11, wherein said sodium ascorbate is present in an amount of at least about 100 mM.

13. The method according to claim 11, wherein said sodium ascorbate is present in an amount of from about 5 to about 500 mM.

14. The method according to claim 13, wherein said sodium ascorbate is present in an amount of from about 10 to about 400 mM.

15. The method according to claim 14, wherein said sodium ascorbate is present in an amount of from about 50 to about 300 mM.

16. The method according to claim 15, wherein said sodium ascorbate is present in an amount of from about 75 to about 200 mM.

17. The method of claim 1, wherein the product comprises an enzyme.

18. The method of claim 17, wherein said enzyme is alpha-galactosidase.

19. The method according to claim 17, wherein said enzyme is trypsin.

20. The method according to claim 17, wherein said enzyme is iduronate-2-sulfatase.

21. The method according to claim 1, wherein said product is a blood component.

22. The method according to claim 21, wherein said blood component is a blood protein.

23. The method according to claim 22, wherein said blood protein is selected from the group consisting of albumin, lipoproteins, complement proteins, globulins, Factor I (fibrinogen), Factor II (prothrombin), Factor III (tissue factor), Factor V (proaccelerin), Factor VI (accelerin), Factor VII (proconvertin, serum prothrombin conversion), Factor VIII (antihemophiliac factor A), Factor IX (antihemophiliac factor B), Factor X (Stuart-Prower factor), Factor XI (plasma thromboplastin antecedent), Factor XII (Hageman factor), Factor XIII (protransglutamidase), von Willebrands factor (vWF), Factor Ia, Factor IIa, Factor IIIa, Factor Va, Factor VIa, Factor VIIa, Factor VIIIa, Factor IXa, Factor Xa, Factor XIa, Factor XIIa, Factor XIIIa, hemoglobin and growth factors.

24. The method according to claim 21, wherein said blood component is a liquid blood component.

25. The method according to claim 24, wherein said liquid blood component is plasma or serum.

26. The method according to claim 1, wherein said product is a proteinaceous material.

27. The method according to claim 1, wherein said product is an immunoglobulin.

28. The method according to claim 27, wherein said immunoglobulin is selected from the group consisting of polyclonal IgA, IgM, IgG and IgE and monoclonal immunoglobulin.

29. The method according to claim 1, wherein said product is a coagulation protein.

30. The method according to claim 29, wherein said coagulation protein is selected from the group consisting of Factor VII, Factor VIII, Factor IX and von Willebrands factor.

31. A product made according to the process of one of claims 1-30.

32. An improved method for lyophilizing or freeze-drying a product, wherein the improvement comprises adding a compound effective to reduce residual solvent content to the product prior to lyophilizing or freeze-drying the product, the amount of said compound added being effective to lower the residual solvent content of said product following lyophilization or freeze-drying.

33. The method according to claim 32, wherein said compound effective to reduce residual solvent content is selected from the group consisting of mannitol, ascorbic acid, sodium ascorbate and methyl ascorbate.

34. The method according to claim 33, wherein said compound effective to reduce residual solvent content is mannitol.

35. The method according to claim 33, wherein said compound effective to reduce residual solvent content is sodium ascorbate.

36. The method according to claim 32, wherein the product has a residual solvent content of less than about 15% following said lyophilization or freeze-drying.

37. The method according to claim 32, wherein the product has a residual solvent content of less than about 10% following said lyophilization or freeze-drying.

38. The method according to claim 32, wherein the product has a residual solvent content of less than about 5% following said lyophilization or freeze-drying.

39. The method according to claim 32, wherein the product has a residual solvent content of less than about 3.0% following said lyophilization or freeze-drying.

40. The method according to claim 32, wherein the product has a residual solvent content of less than about 2.0% following said lyophilization or freeze-drying.

41. The method according to claim 32, wherein the product has a residual solvent content of less than about 1.0% following said lyophilization or freeze-drying.

42. The method according to claim 32, wherein the product has a residual solvent content of less than about 0.5% following said lyophilization or freeze-drying.

43. The method according to claim 32, wherein the product has a residual solvent content of less than about 0.2% following said lyophilization or freeze-drying.

44. The method according to claim 32, wherein the product has a residual solvent content of less than about 0.08% following said lyophilization or freeze-drying.

45. The method of claim 32, wherein the product comprises an enzyme.

46. The method of claim 45, wherein said enzyme is alpha-galactosidase.

47. The method according to claim 45, wherein said enzyme is trypsin.

48. The method according to claim 45, wherein said enzyme is iduronate-2-sulfatase.

49. The method according to claim 32, wherein said product is a blood component.

50. The method according to claim 49, wherein said blood component is a blood protein.

51. The method according to claim 50, wherein said blood protein is selected from the group consisting of albumin, lipoproteins, complement proteins, globulins, Factor I (fibrinogen), Factor II (prothrombin), Factor III (tissue factor), Factor V (proaccelerin), Factor VI (accelerin), Factor VII (proconvertin, serum prothrombin conversion), Factor VIII (antihemophiliac factor A), Factor IX (antihemophiliac factor B), Factor X (Stuart-Prower factor), Factor XI (plasma thromboplastin antecedent), Factor XII (Hageman factor), Factor XIII (protransglutamidase), von Willebrands factor (vWF), Factor Ia, Factor IIa, Factor IIIa, Factor Va, Factor VIa, Factor VIIa, Factor VIIIa, Factor IXa, Factor Xa, Factor XIa, Factor XIIa, Factor XIIIa, hemoglobin and growth factors.

52. The method according to claim 49, wherein said blood component is a liquid blood component.

53. The method according to claim 52, wherein said liquid blood component is plasma or serum.

54. The method according to claim 32, wherein said product is a proteinaceous material.

55. The method according to claim 32, wherein said product is an immunoglobulin.

56. The method according to claim 55, wherein said immunoglobulin is selected from the group consisting of IgA, IgM, IgG and IgE and monoclonal immunoglobulins.

57. The method according to claim 32, wherein said product is a coagulation protein.

58. The method according to claim 57, wherein said coagulation protein is selected from the group consisting of Factor VII, Factor VIII, Factor IX and von Willebrands factor.

59. The method according to claim 32, wherein said compound effective to reduce residual solvent content comprises mannitol and sodium ascorbate.

60. A product made according to the process of one of claims 32-59.

61. A method for prophylaxis or treatment of a disease or infection in a mammal comprising administering to a mammal in need thereof an effective amount of a product made according to the method of one of claims 1-30.

62. The method according to claim 61, wherein said mammal is a human.

63. A method for prophylaxis or treatment of a disease or infection in a mammal comprising administering to a mammal in need thereof an effective amount of a product made according to the method of one of claims 32-59.

64. The method according to claim 63, wherein said mammal is a human.

65. The method according to one of claims 1-30 and 32-59, wherein said solvent is water.
